(19) United States
(12) Patent Application Publication
(10) Pub. No.: US 2016/0029881 A1
(43) Pub. Date: Feb. 4, 2016

(54) LENS CLEANING INSTRUMENT FOR SURGICAL PROCEDURES PERFORMED IN AN ENCLOSED CAVITY

(71) Applicant: HEALTH RESEARCH, INC.,, Buffalo, NY (US)

(72) Inventors: Khurshid GURU, East Amherst, NY (US); Ashirwad CHOWRIAPPA, Buffalo, NY (US)

(21) Appl. No.: 14/776,092

(22) PCT Filed: Mar. 14, 2014
(86) PCT No.: PCT/US14/29718

§ 371 Date: Sep. 14, 2015

Related U.S. Application Data
(60) Provisional application No. 61/798,179, filed on Mar. 15, 2013.

(51) Int. Cl. A61B 1/12

(52) U.S. Cl. CPC A61B 1/126; A61B 2019/343

(57) ABSTRACT

A device for cleaning a lens is disclosed. The device includes a shaft, a head having multiple prongs, a cleaning material disposed between the prongs, and a handle extending from the shaft. The handle has an actuator in communication with the prongs to move the prongs from a collapsed state to an expanded state. Also disclosed is a cartridge for use with the device. The cartridge includes a cartridge head configured for removable attachment to the device, two or more prongs, and a wipe. The head has a fixed pivot pin and a slide pin movable by the actuator. Each prong has a pivot aperture configured to engage with the pivot pin and a slot aperture configured to engage with the slide pin such that the corresponding prong will be urged to rotate about the pivot pin when the slide pin is moved by the actuator.

### CROSS-REFERENCE TO RELATED APPLICATIONS

This application claims priority to U.S. Provisional Application No. 61/798,179, filed on Mar. 15, 2013, now pending, the disclosure of which is incorporated by reference.

### FIELD OF THE INVENTION

The invention relates to a lens cleaning instrument, namely a lens cleaning instrument for surgical procedures performed in an enclosed cavity. For example, the lens cleaning instrument may be used during laparoscopy and/or robot assisted surgery.

### BACKGROUND OF THE INVENTION

Laparoscopes are surgical instruments through which anatomical structures in the abdomen and pelvis can be viewed by a surgeon. One of the limitations of conventional laparoscopes is that the laparoscope lens frequently contacts tissue and is obscured by blood and other matter during a laparoscopic procedure. This adversely affects the quality of the image viewed by the surgeon on the display unit.

Currently, when a lens becomes obscured, the camera must be removed from the body to be cleaned. Removing the tool is time intensive and disrupts the flow of the surgery being performed. Furthermore, the temperature of the lens changes as it moves from a cleaning solution, to the ambient air, and finally to the body. This rapid temperature change may cause the lens to fog. If the lens fogs, the camera must be removed and cleaned once more.

Prior designs involved a cleaning instrument that remained in the cavity during surgery. These cleaning instruments are difficult to locate within the body and are at risk of being lost within the cavity. Furthermore, because the cleaning instruments of the prior design would often become dirty by virtue of resting in the cavity where the surgery is being performed.

### BRIEF SUMMARY OF THE INVENTION

The invention may be described as a device for cleaning a lens. The device comprises a shaft, a head, a cleaning material, and a handle.

The shaft may be hollow. In one embodiment, the device further comprises a plunger in mechanical communication with the head.

The head is attached to the shaft and has two or more prongs. The head may be removable attached to the shaft. Each prong may have a pivot aperture and a slot aperture. The head may further comprise a pivot pin and a slide pin. The pivot pin is in a fixed position relative to the head. The slide pin is configured to be moved when the actuator is operated. The pivot pin is disposed in the pivot aperture of each prong and the slide pin is disposed in the slot aperture of each prong such that each prong is urged to rotate about the pivot pin when the slide pin is moved.

The cleaning material is disposed between the two or more prongs. The handle extends from the shaft. The handle has an actuator in mechanical communication with the prongs of the head. The actuator is operable to move the two or more prongs from a collapsed state to an expanded state. The actuator may be a mechanical switch. In one embodiment, the device further comprises an articulating joint between the shaft and the head.

The invention may also be described as a cartridge for use with a device for cleaning a lens. The cartridge comprises a cartridge head, two or more prongs, and a wipe. The cartridge head is configured for removable attachment to the device. The cartridge head has a pivot pin in a fixed position and a slide pin configured to be moved by the actuator of the device when the cartridge is attached to the device. Each prong has a pivot aperture configured to engage with the pivot pin of the cartridge head, and a slot aperture configured to engage with the slide pin of the cartridge head such that the corresponding prong will be urged to rotate about the pivot pin when the slide pin is moved by the actuator. The wipe is disposed between the two or more prongs.

### DESCRIPTION OF THE DRAWINGS

For a fuller understanding of the nature and objects of the invention, reference should be made to the following detailed description taken in conjunction with the accompanying drawings, in which:

FIG. 1 is a diagram of one embodiment of the present invention. illustrating handle 1, shaft 2, head 3, and cleaning cartridge 4;

FIG. 2a is a magnified view of the head 3 of one embodiment of the present invention in the closed position;

FIG. 2b is a magnified view of the head 3 of one embodiment of the present invention in the closed position and further illustrating the range of motion when opened;

FIG. 3a is a magnified view of the head 3 of one embodiment of the present invention in the open position;

FIG. 3b is a magnified view of the head 3 of one embodiment of the present invention in the open position with a cleaning cloth;

FIG. 4 is an illustration of one embodiment of the present invention depicting the operation of the device, including opening and closing the cleaning head 3 using a switch on the handle 1;

FIG. 5 is a wireframe drawing of one embodiment of the present invention showing the head 3 in an open position;

FIG. 6 is an illustration of an isometric and side view of one embodiment of the present invention depicting a cartridge rack containing cleaning cartridges 4 in a cartridge;

FIG. 7 is an illustration of one embodiment of the present invention showing the head 3 and the cartridge mechanism 4. The cartridge head 3 is loaded on the instrument head;

FIGS. 8a, 8b, 8c, and 8d are wireframe drawings of one embodiment of the present invention showing an exploded view of a head;

FIG. 9 is a schematic of one embodiment of the present invention showing a sub-element of a head;

FIG. 10 is a schematic of one embodiment of the present invention showing a sub-element of a head;

FIG. 11 is a schematic of one embodiment of the present invention showing a sub-element of a head;

FIG. 12 is a schematic of one embodiment of the present invention showing a shaft;

FIG. 13 is a schematic of one embodiment of the present invention showing a sub-element of a head;

FIG. 14 is a schematic of one embodiment of the present invention showing a sub-element of a head; and

FIGS. 15a, 15c, and 15e are images from a camera immersed in 20%, 60% and 100% saturated solutions, respectively. FIGS. 15b, 15d, and 15f are images from the same camera after 1-3 wipes from an embodiment of the present invention.

### DETAILED DESCRIPTION OF THE INVENTION

The lens cleaning instrument of the present invention is capable of improving clarity of vision of an operative field during a surgical procedure.

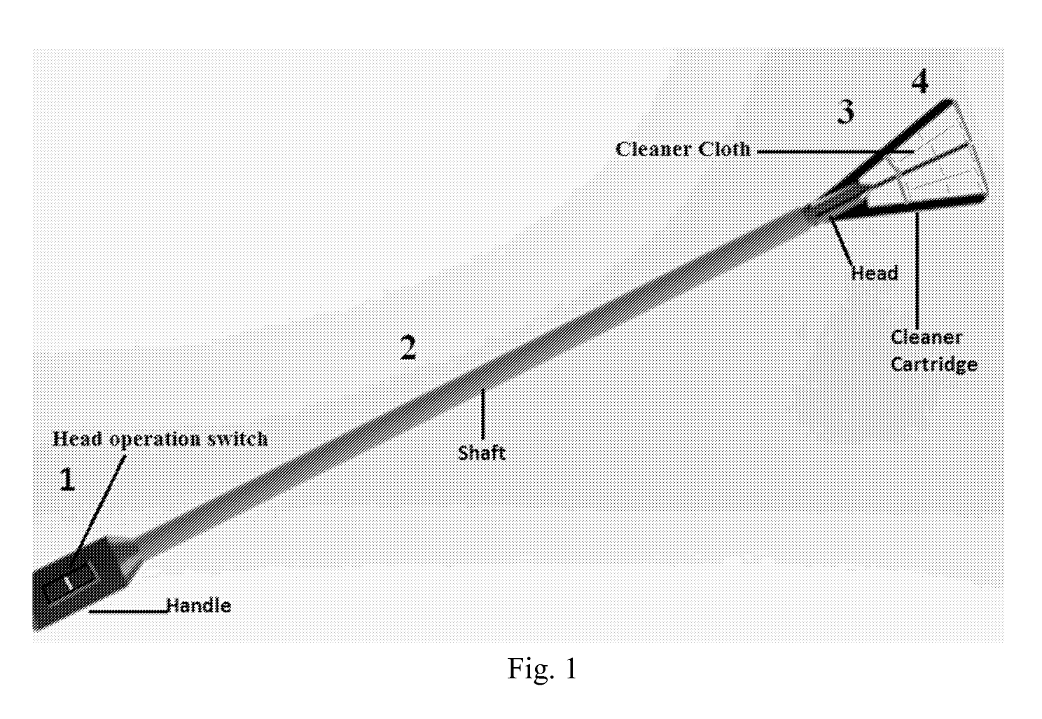

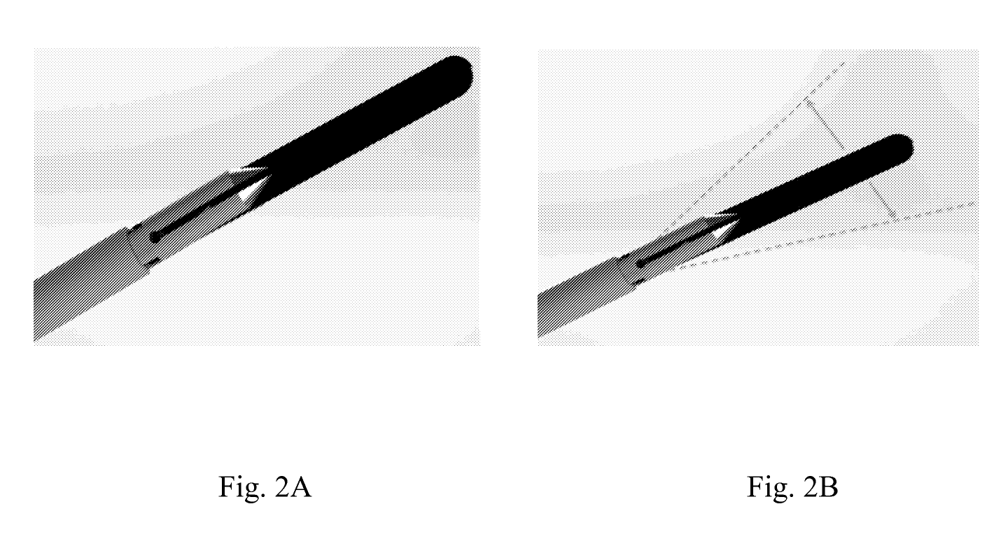

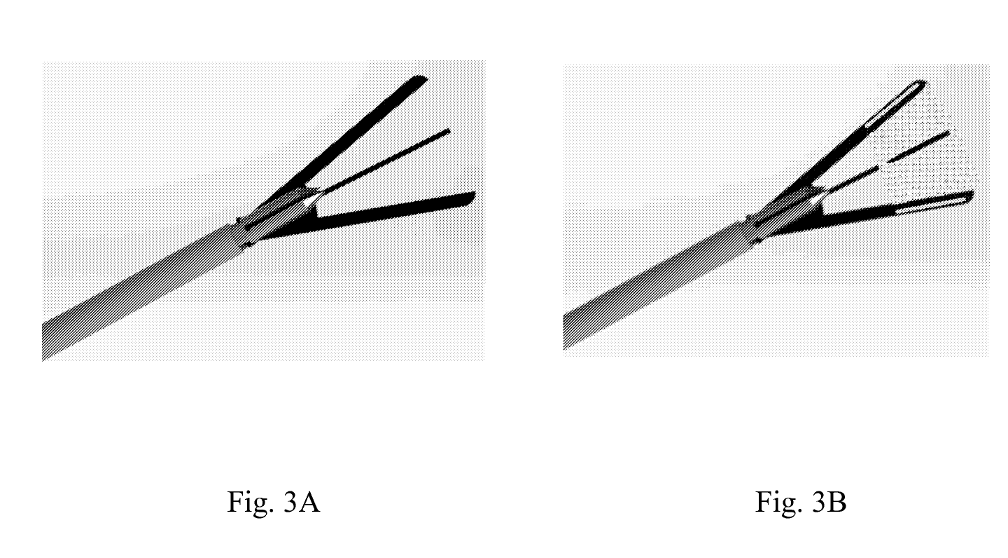

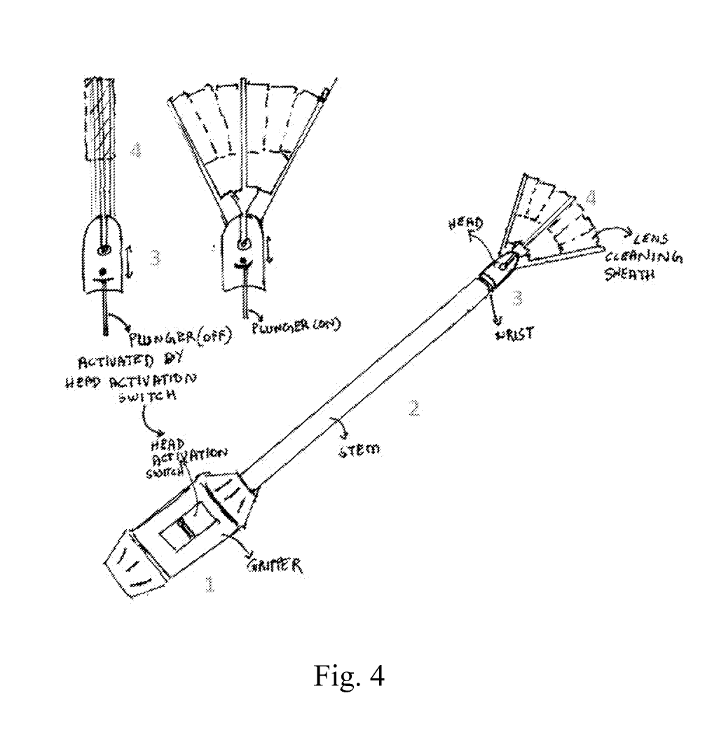

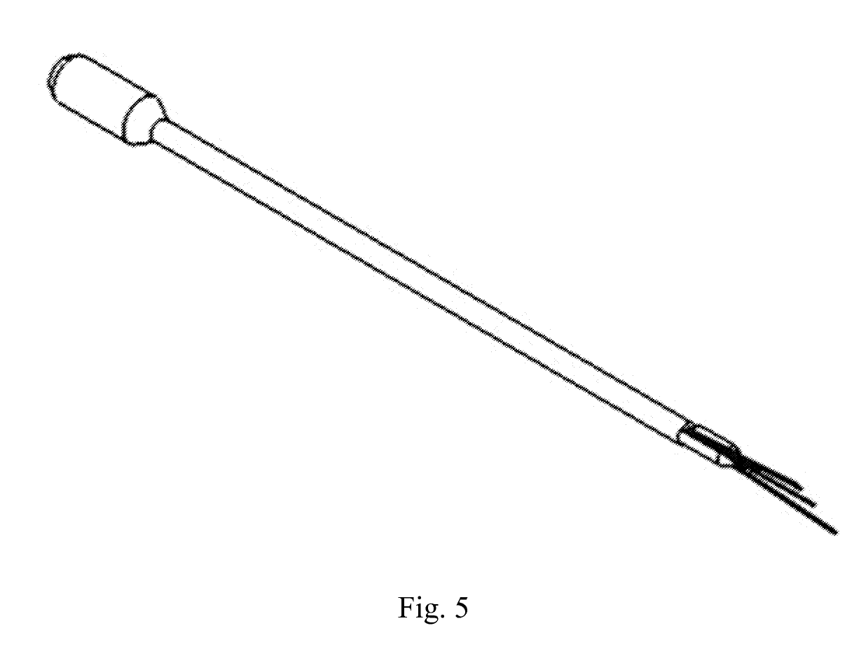

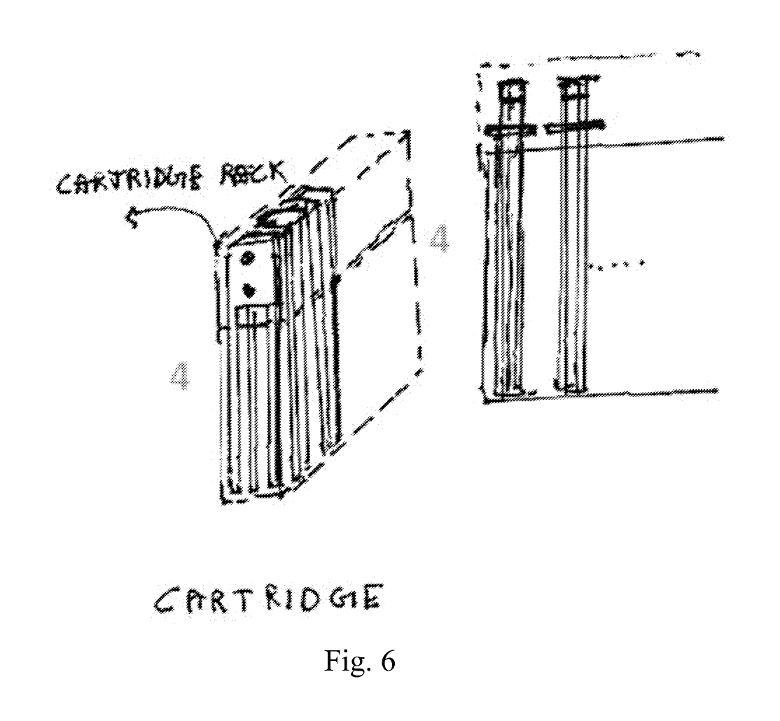

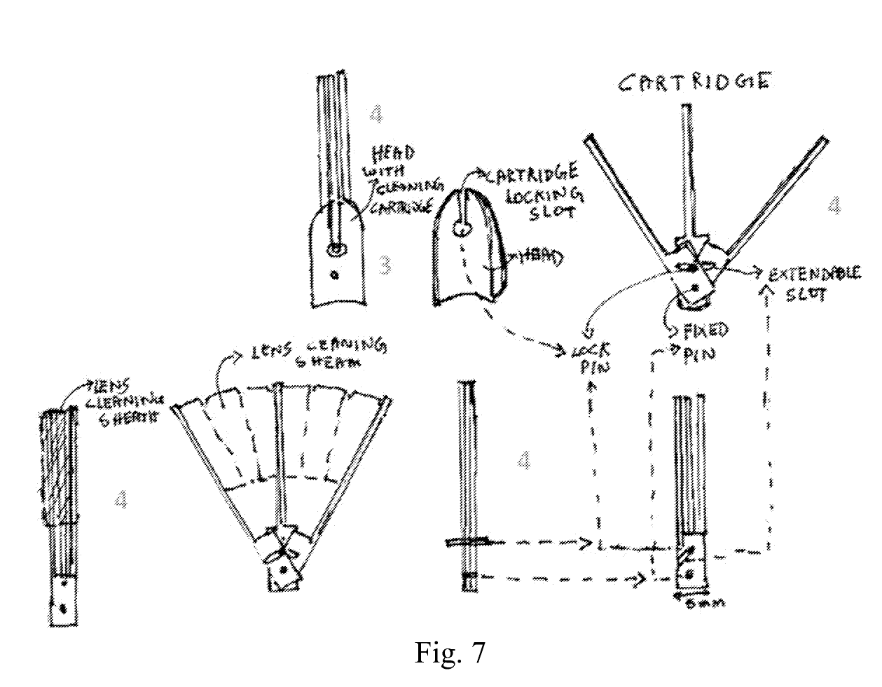

The present invention may be embodied as a laproscopic instrument 10 for cleaning a lens used in minimally invasive surgical procedures, such as that pictured in FIG. 1.

The instrument 10 comprises a handle 12, a head 14, and a shaft 16. The handle 12 comprises an actuator 13, which may be, for example, a switch, a lever, a button, or any other type of actuator 13 known in the art.

The head 14 has two or more prongs 20 and a wipe 28 disposed between the two or more prongs 20. Each prong 20 is attached to the head 14 by a pivot pin 18, and each prong 20 is rotatable about the pivot pin 18. The head 14 further comprises a slide pin 19 which is in mechanical communication with the actuator 13 of the handle 12. In some embodiments, the head 14 has a slot 29 which causes the slide pin 19 to be movable in only one dimension (e.g., translated along a path which is substantially parallel to a longitudinal axis of the instrument 10). In this way, when the actuator 13 is used by an operator (e.g., a surgeon, nurse, etc.), the slide pin 19 is moved (i.e., translated-extended, etc.) in the slot 29. The slide pin 19 is disposed in a slot aperture 24 of each prong 20 such that the slide pin 19 is movable within the slot aperture 24.

In another embodiment, the prongs 20 may be attached to one another by the pivot pin 18—a prong assembly. The prongs 20 may be attached to the head 14 by the slide pin 19. The prongs 20 may be removably attached to the head 14. A plunger may be in mechanical communication with the actuator 13 of the handle 12, and the plunger may act on the prong assembly (e.g., pushing) to cause the prong assembly to move relative to the head 14. This motion of the prong assembly relative to the head 14 causes the slide pin 19 to move in the slot apertures 24 of the prongs 20, thereby urging the prong assembly to expand or collapse as further described below. Similarly, the plunger may act on the prong assembly (e.g., pulling) to cause the prong assembly to collapse.

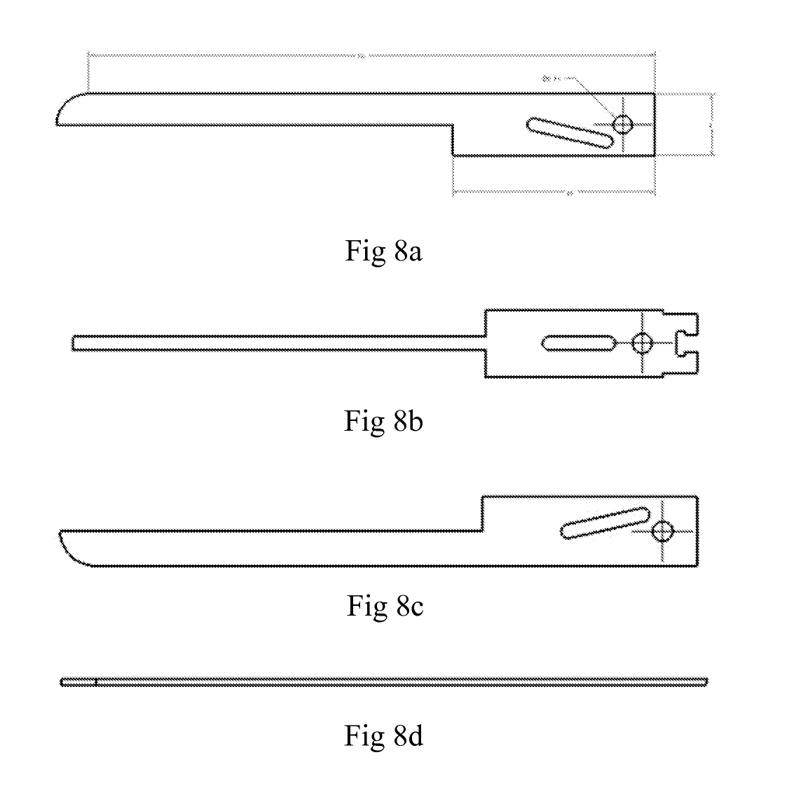

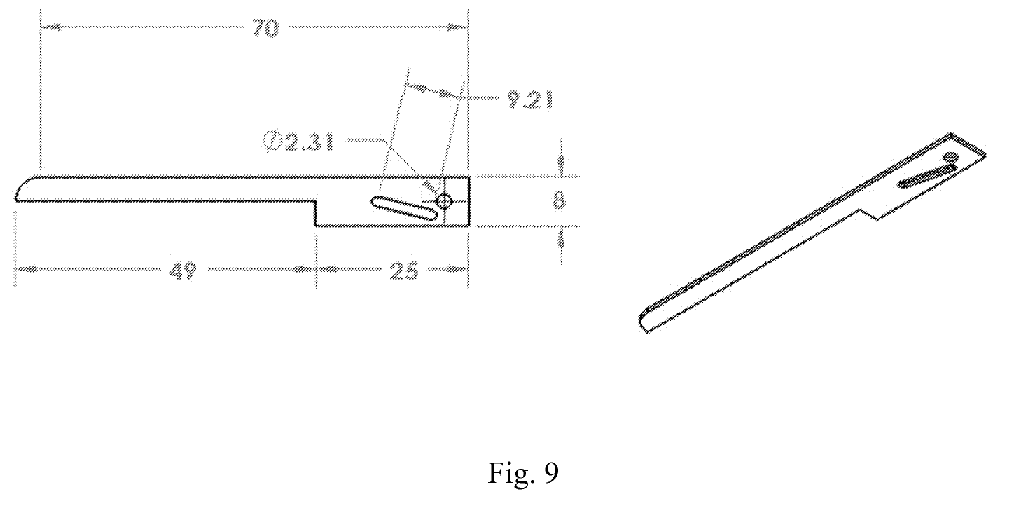

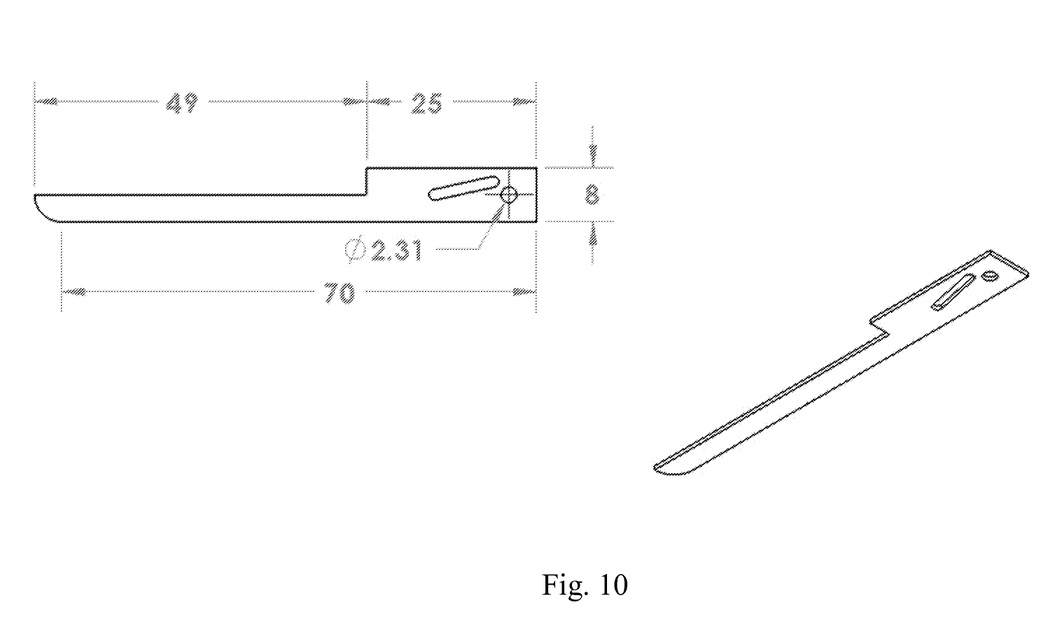

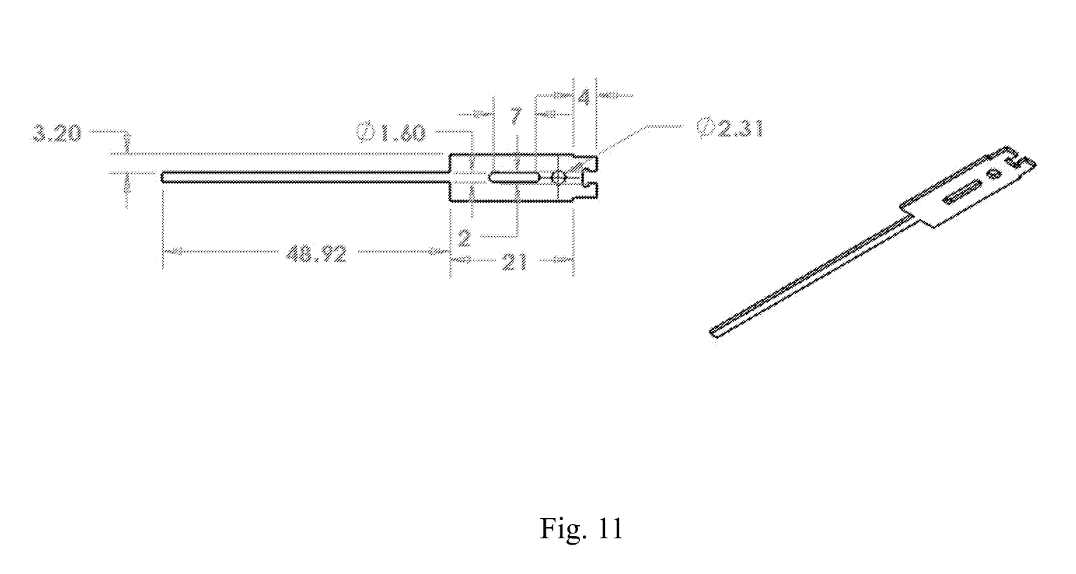

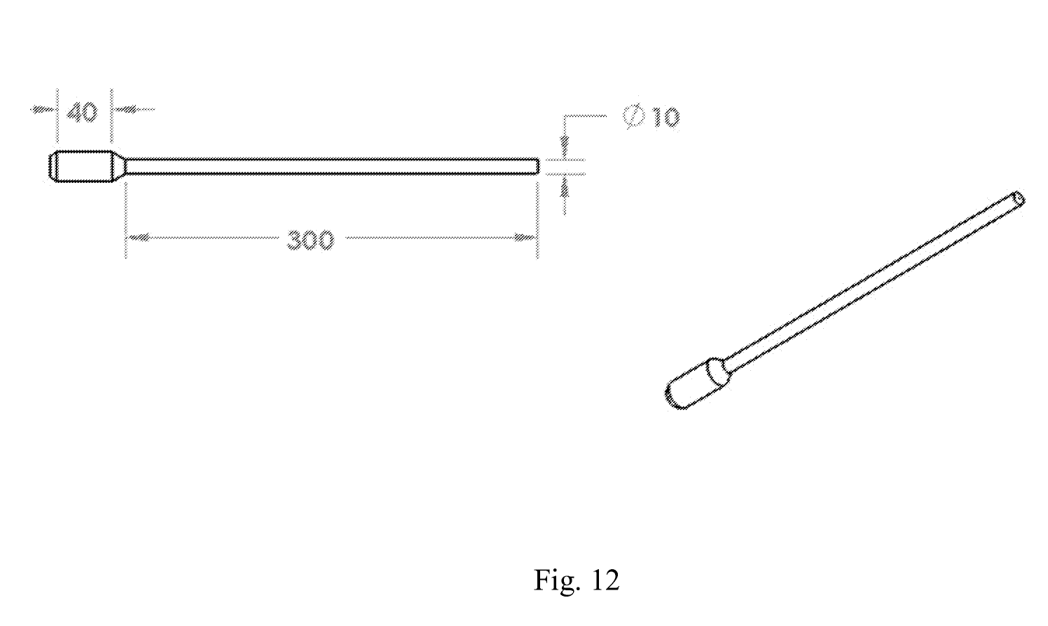

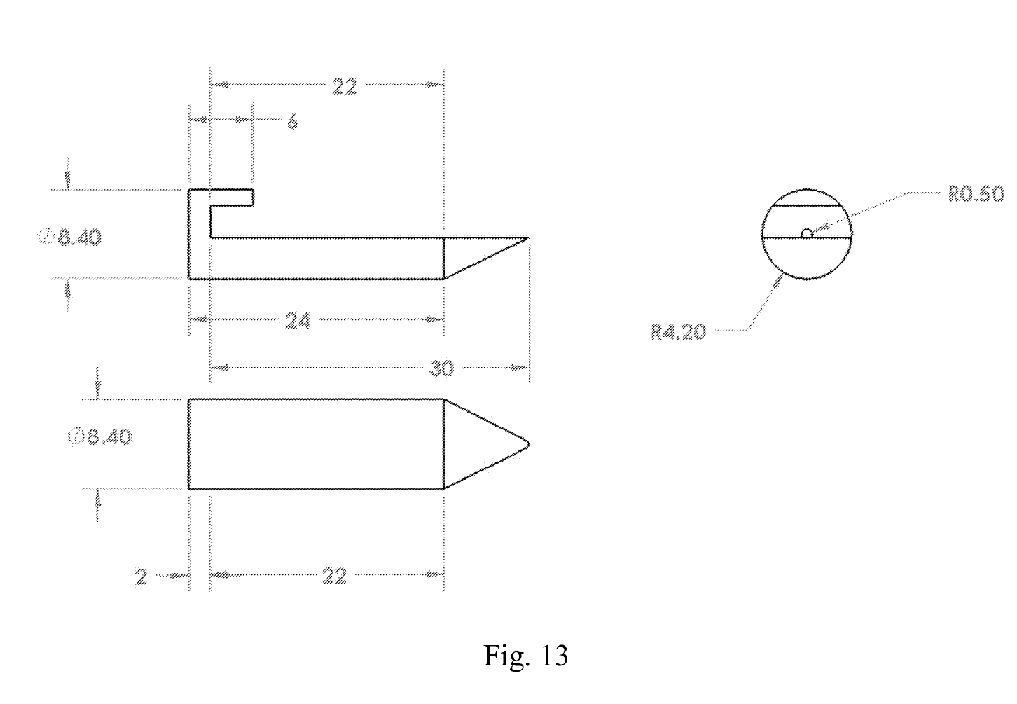

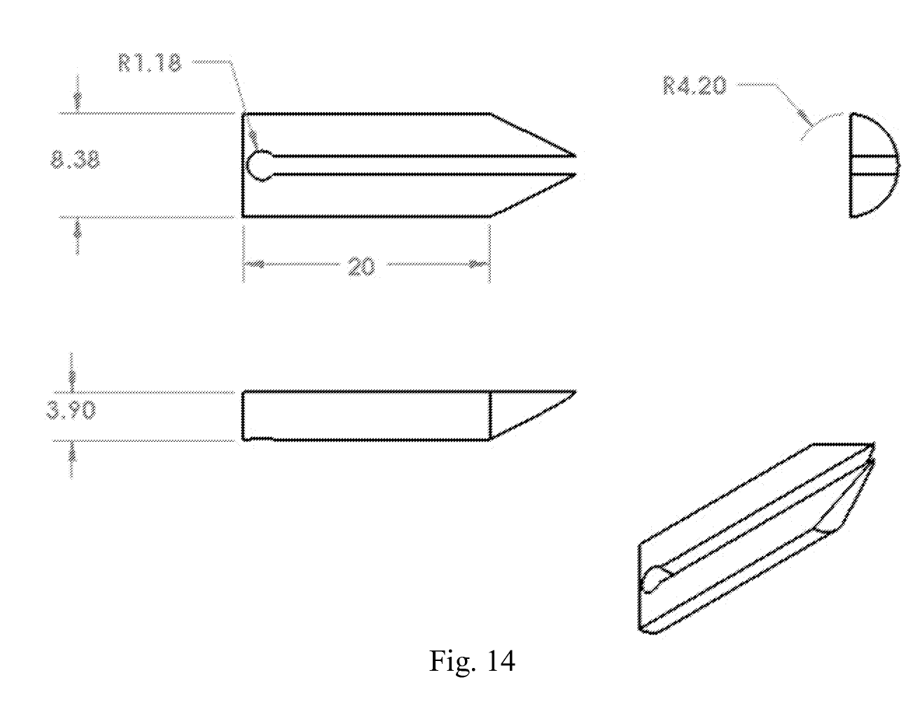

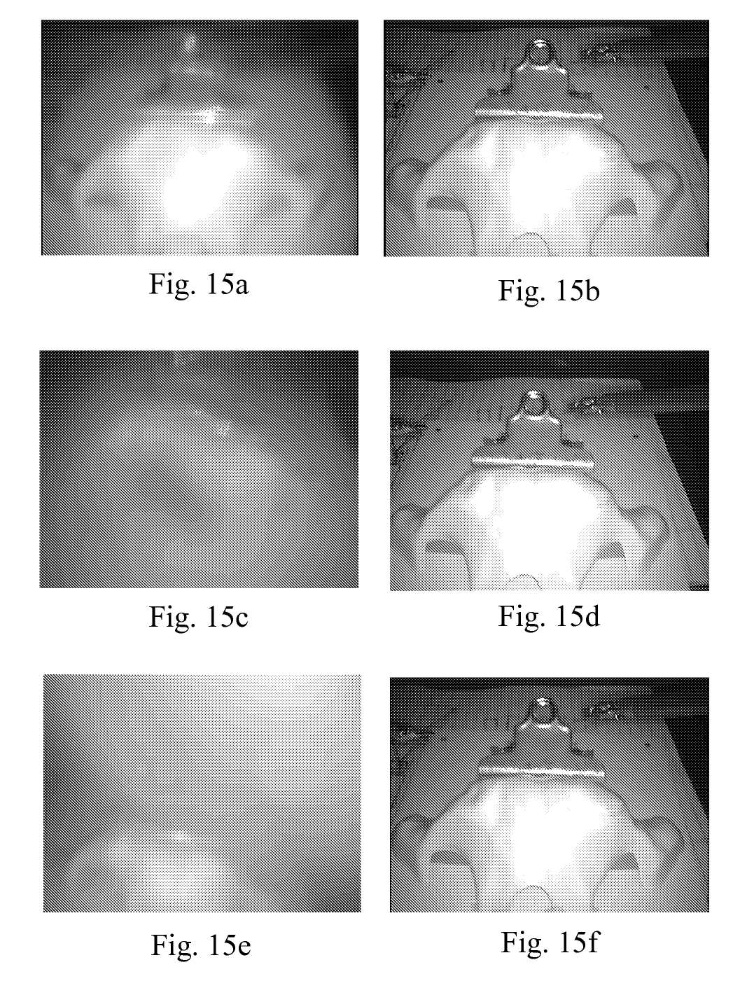

FIGS. 8A-8C show three exemplary prongs 30, 32, 34 suitable for use in an embodiment of the present invention. Prong 30 is shown with pivot aperture 36 (through which the pivot pin 18 is disposed) and slot aperture 31 through which the slide pin 19 is disposed. Slot aperture 31 is configured to urge the prong 30 to rotate about the pivot aperture 36 when the slide pin 19 is moved within the slot aperture 31—slide pin 19 is primarily able to translate left-right relative to the diagram, thereby causing the prong 30 to rotate due to the angle of the slot aperture 31.

Similarly, prong 34 is shown with slot aperture 35 causing a rotation about the pivot aperture in an opposite direction from that of prong 30 when the prongs 30, 34, are urged to move by slide pin 19. In FIG. 8B, a prong is depicted having a slot aperture 33 with no angle such that the prong 32 will generally not be caused to rotate about pivot aperture 36 when the slide pin 19 is moved. In this manner, when the prongs 30, 32, 34 are used in a head 14 and engaged with a slide pin 19, the rotatable prongs 30, 32, 34 will be urged to rotate between an expanded state and a collapsed state when the slide pin 19 is moved (by use of the actuator 13).

A wipe 28 is disposed between the prongs 20 of the instrument 10. In this manner, when the prongs 20 are in the expanded state, the wipe 28 is “deployed” such that it may be used to clean a lens of a camera.

The shaft 16 is configured to connect the handle 12 and the head 14. The shaft may be hollow such that a mechanical linkage within the shaft 16 can be used to operably connect the prongs 20 of the head 14 with the actuator 13 of the handle 12.

In use, the head 14 of an instrument 10 may be inserted into a surgical field through an incision with the prongs 20 in the collapsed state. In this way, the overall cross-sectional area of the head 14—with respect to a longitudinal axis—is minimized and able to pass through an incision. Once the head 14 is at a desired position within the surgical field (e.g., near the lens of the camera, the actuator 13 may be used by the operator to move the slide pin 19 thereby urging the prongs 19 to an expanded state and deploying the wipe 28. The operator may then manipulate the instrument in order to clean the lens of the camera using the wipe 28. The camera may be used by the operator to move the instrument appropriately for cleaning.

When the operator is ready for removal of the instrument 10, the actuator 13 may be used to move the slide pin 19 in the opposite direction, thereby urging the prongs 20 into the collapsed state such that the head 14 may be removed through the incision.

It should be noted that the wipe 28 may be sized such that the surface area allows multiple uses before the wipe 28 is soiled and can no longer be used for cleaning. Additionally, the instrument 10 may allow a second side of the wipe 28 to be utilized by, for example, rotating the instrument 10 about the shaft 16.

The head 14 may be removable from the shaft 16. In such embodiments, the head 14 can be considered disposable and modular (i.e., a disposable cartridge). In this manner, the cartridge may be attached to an instrument, used to clean a lens (one or more times), and removed from the instrument such that an unused cartridge may be attached to the same instrument.

In other embodiments, the prongs 20 and the wipe 28 may be removable from the head 14, and in such embodiments, the prongs 20 and wipe 28 may be considered a cartridge. As such, the prongs 20 may be pivotably connected to one another by a pivot pin 18, which may also make up a portion of the cartridge.

In one embodiment, a lens cleaning instrument which is suitable for reducing the time required to clean the lens of a camera during a minimal invasive surgical procedure is disclosed. An illustrative embodiment of the laparoscopic lens cleaner includes an instrument gripper/handle, stem/shaft, and a head, which may be a replaceable cleaning cartridge (see FIG. 1). The cartridges can be detached from the instrument and replaced once soiled.

In another embodiment, a lens cleaning instrument according to the present invention comprises a shaft, a head, a cleaning material, and a handle.

The shaft may be hollow to allow for mechanical linkages between the head and the handle. The shaft may also be flexible, such that the position and movement of the shaft may be better controlled by the user. The shaft may be configured to accept a head through friction-fit, screw, or other means. In some embodiments, the head may be removably attached to the shaft, while in other embodiments, the head is permanently attached.

The head has two or more prongs extending from the head. In one embodiment, there are three prongs. In another embodiment, there are four prongs positioned at right angles from each other.

A cleaning material is disposed between the two or more prongs. The cleaning material may be attached to the prongs with an adhesive, tied to the prongs, or otherwise affixed to the prongs. The material may also be threaded through the prongs. The cleaning material may be made from any flexible material known for such uses, such as, for example, a woven fabric. The material may be double sided in order to increase the area that can be used to clean the lens of a camera.

The handle may be ergonomic or textured to increase grip and/or comfort. The handle contains a controller (i.e., an actuator) which is in communication with the head. The controller may be a mechanical device such as a switch, button, trigger, or joystick. Manipulating the actuator manipulates the prongs of the head. This manipulation may be direct or indirect. For example, the actuator may be a switch. When the switch moves, it pulls a cable which is attached to the prongs of the head. When a force is applied to the prongs through the cable, the prongs pivot and open. Likewise, when the cable is released, the prongs may be biased to return to a closed position.

In one embodiment, each prong has a pivot aperture and a slot. In this embodiment, the head further comprises a fixed pin positioned in the pivot aperture of each of the prongs, and a slide pin positioned in the slot of each of the prongs. Moving the actuator moves the slide pin thereby manipulating the two or more prongs of the head.

In another embodiment, the head may be attached to the shaft using a articulating joint (sometimes referred to as a “wrist joint”). A wrist joint allows the user to control the angle of the head with reference to the shaft.

As mentioned above, the head of the present invention may be removably attached to the shaft. In these embodiments, the heads may be packaged as cartridges that allows quick detachment of a used head and quick attachment of a new head. For example, the shaft may be configured to receive a new head by having a groove configured to create a friction fit with the pivot pin of the head. Multiple cartridges may be stored in a holder such as a rack.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof

What is claimed is:

1. A device for cleaning a lens, comprising:
a shaft;
a head attached to the shaft, the head having two or more prongs;
a cleaning material disposed between the two or more prongs; and
a handle extending from the shaft having a actuator in mechanical communication with the prongs of the head, wherein the actuator is operable to move the two or more prongs from a collapsed state to an expanded state.

2. The device of claim 1, wherein the head is removably attached to the shaft.

3. The device of claim 1, wherein the actuator is a mechanical switch.

4. The device of claim 1, wherein the shaft is hollow and the device further comprises a plunger in mechanical communication with the head.

5. The device of claim 4, wherein each prong has a pivot aperture and a slot aperture.

6. The device of claim 5, wherein the head further comprises a pivot pin in a fixed position relative to the head and a slide pin configured to be moved when the actuator is operated; and wherein the pivot pin is disposed in the pivot aperture of each prong and the slide pin is disposed in the slot aperture of each prong such that each prong is urged to rotate about the pivot pin when the slide pin is moved.

7. The device of claim 1, wherein the device further comprises an articulating joint between the shaft and the head.

8. A cartridge for use with a device for cleaning a lens, the device having an actuator, the cartridge, comprising:
a cartridge head configured for removable attachment to the device, the cartridge head having a pivot pin in a fixed position and a slide pin configured to be moved by the actuator of the device when the cartridge is attached to the device;
two or more prongs, each prong having a pivot aperture configured to engage with the pivot pin of the cartridge head, and a slot aperture configured to engage with the slide pin of the cartridge head such that the corresponding prong will be urged to rotate about the pivot pin when the slide pin is moved by the actuator;
a wipe disposed between the two or more prongs.
